(19) United States
(12) Patent Application Publication
(10) Pub. No.: US 2008/0175831 A1
(43) Pub. Date: Jul. 24, 2008

(54) NEUROPROTECTION BY BLOOD FLOW STABILIZATION

(76) Inventors: Stephen L. Macknik, Anthem, AZ (US); Susana Martinez-Conde, Anthem, AZ (US)

Correspondence Address: WELSH & KATZ, LTD, 120 S RIVERSIDE PLAZA, 22ND FLOOR, CHICAGO, IL, 60606, US

(21) Appl. No.: 11/961,835

(22) Filed: Dec. 20, 2007

Related U.S. Application Data
(60) Provisional application No. 60/876,354, filed on Dec. 21, 2006.
(60) Provisional application No. 60/977,501, filed on Oct. 4, 2007.

(51) Int. Cl. A61K 38/45; A61K 31/727; A61K 31/60; A61K 31/04; A61K 31/5575; A61K 31/4164; A61K 31/4375; A61K 31/19; A61P 3/10; A61P 25/28; A61P 7/02

(52) U.S. Cl. 424/94.5; 514/56; 514/165; 514/569; 514/250; 514/742; 514/385; 514/573

(57) ABSTRACT

A treatment is disclosed for alleviation or prevention of abnormal blood flow to various organs such as the eye, brain, kidneys, heart, feet and other tissues of organs with fine vascular networks that can lead to neurodegeneration as is seen in wet age-related macular degeneration (AMD), epilepsy and diabetes, in which an effective amount of a blood flow regulatory drug is administered to a subject in need of it. Illustrative blood flow regulatory drugs include anticoagulants and vasodilators, and their mixtures.

### CROSS-REFERENCE TO RELATED APPLICATION

This application claims benefit of provisional application Ser. No. 60/876,354 that was filed on Dec. 21, 2006, and of provisional application Ser. No. 60/977,501 that was filed on Oct. 4, 2007, whose disclosures are incorporated by reference.

### TECHNICAL FIELD

The present invention relates to the neurotoxicity caused by abnormal blood flow, and more particularly the alleviation or prevention of abnormal blood flow to various organs such as the eye and brain that can lead to neurodegeneration.

### BACKGROUND ART

Abnormal blood flow can cause either hypoperfusion or hyperperfusion or both, and can lead to swelling and/or oxygen starvation within various organs such as the retina and brain as can happen during an epileptic seizure. It is believed that there exists a strong quantitative link between seizures and pathological blood flow.

Although it has been established that ictal episodes (e.g., seizures) modulate blood flow from resting levels [Penfield et al., Epilepsy and the functional anatomy of the human brain, London, Churchill 1954; Haglund et al. (1992) Nature 358:668-671; Haglund et al. (1998) Determination of seizure propagation pathways by inhibitory-like surround in primate visual cortex, Society for Neuroscience 28th Annual Meeting, Los Angeles, Calif., Society for Neuroscience Abstracts; Schwartz et al. (2001) Nat Med 7(9):1063-1067; Hirase, H., J. Creso, et al. (2004) Neuroscience 128(1):209-16; Hirase et al. (2004)Glia 46(1):95-100; Joo et al. (2004) Epilepsia 45(6):686-689; Suh et al. (2005) J. Neuroscience 25(1):68-77; and Tae, W. S., E. Y. Joo, et al. (2005) Neuroimage 24(1):101-110], no direct comparison between inter-ictal and ictal capillary blood flow and normal flow in wild-type normals has been established previously. Therefore it is not known if ictal blood flow at the level of individual capillaries truly leads to transient ischemia and/or hyperemia outside of the range of normal blood flow. This gap in knowledge has prevented the field from moving forward with research on neuroprotective therapies to mitigate the chronic neural damage caused by ictal blood flow.

In the United States, approximately 2 million people are afflicted by epilepsy [Hauser, (1992) Epilepsy Res Suppl 5:25-28] Severe ictal episodes, such as repetitive generalized seizures in children [Schulz et al., (2001) Neurology 57(2):318-320] and adults [Clark et al., (1903) Am. J. Insanity 60:291-306; Briellmann et al., (2001) Neurology 57(2):315-317] lead to brain tissue damage in approximately two-thirds or more of patients of temporal lobe epilepsy [Cavanagh et al., (1956) Br. Med. J. 2:1403-1407; Mathern et al., (2002) Prog Brain Res 135:237-251; Thom et al. (2002) J Neuropathol Exp Neurol 61(6): 510-519].

Ictal brain damage has been correlated with cognitive and behavioral deficits [Fuerst et al., (2001) Neurology 57(2):184-188] such as memory loss [Kotloski et al., (2002) Prog Brain Res 135:95-110], psychosis [Briellmann et al., (2000) Neurology 55(7):1027-1030], and even death [Noe et al., (2005). Drugs Today (Barc) 41(4):257-266]. Anticonvulsant drugs administered to suppress episodes of status epilepticus have a long-term neuroprotective effect [Ben-Ari et al., (1979) Brain Res 165(2):362-365; Sutula et al., (1992) J Neurosci 12(11):4173-4187]. In the last decade, evidence of cumulative damage done by brief seizures has been reported to lead to permanent brain damage [Bengzon et al., (1997) Proc Natl Acad Sci USA 94(19):10432-10437; Pretel et al., (1997) Acta Histochem 99(1):71-79; Zhang et al., (1998) Brain Res Mol Brain Res 55(2):198-208; Briellmann et al. (2000) Neurology 55(10):1479-1485; Fuerst et al., (2001) Neurology 57(2):184-188; Kotloski et al., (2002) Prog Brain Res 135:95-110].

Ictal excitotoxicity has a well established link to ischemia and edema, which are associated with epilepsy-associated damage [Spielmeyer (1927) Z. Ges. Neurol. Psychiatrie 109:501-520; Bouilleret et al. (2000), Brain Res 852(2):255-262; Arundine et al., (2003) Cell Calcium 34(4-5):325-337; Fabene et al., (2003) Neuroimage 18(2):375-389; Moldrich et al. (2003) Eur J Pharmacol 476(1-2):3-16; Gee et al., (2005) Cell Mol Life Sci 62(10):1120-1130]. However, the damage and neuronal morphological changes caused by excitotoxicity are also associated with pathological blood flow, such as the ischemia and/or hyperemia seen in stroke [Zhang et al., (2005) J Neurosci 25(22):5333-5338]. Just as abnormal blood flow in strokes can lead to neural damage, seizure-related abnormal blood flow (such as vasospasms causing both ischemia and hyperemia) are thought to also contribute to neural damage (above and beyond the damage done by excitotoxicity) [Pfleger, (1880)Alg. Z. Psychiatrie 36:359-365; Spielmeyer, (1930) Arch. Neurol. Psychiatry 23:869-875; Penfield, W. and H. Jasper (1954) Epilepsy and the functional anatomy of the human brain. London, Churchill; Ingvar, (1986) Ann NY Acad Sci 462:194-206; Ingvar et al., (1981) Acta Physiol Scand 111(2): 05-12; Ingvar et al., (1983) Acta Neurol Scand 68(3):129-144; Ingvar et al., (1984) Epilepsia 25(2):191-204].

The link between epilepsy and neuronal damage was first noticed almost 200 years ago [Bouchet et al., (1825) Arch Gén Med 9:510-542]. Since then, it has been established that seizures trigger a cascade of biochemical, anatomical, and functional changes that can, in some cases, lead to cell death and apoptosis [Cavanagh, et al., (1956). Br Med J 2:1403-1407; Meldrum, (2002) Prog Brain Res 135:3-11]. Neural events following seizures include neural injury, neurogenesis, gliosis, sprouting, and functional changes; it is currently a hot topic of debate whether these events occur in series as a cascade, or in parallel, or in some combination of serial and parallel processes [Cole et al., (2002) Prog Brain Res 135:13-23].

These various events are in general investigated in the context of primary excitotoxic mitochondrial damage resulting from calcium overload during ictal burst discharge [Meldrum et al., (1973) Arch Neurol 29(2):82-87; Olney et al., (1983) Brain Res Bull 10(5): 699-712; Olney et al., (1986) Adv Neurol 44:857-877; Sloviter, (1983) Brain Res Bull 10(5):675-697; Sloviter, (1986) Adv Exp Med Biol 203:659-671]. However, some of the earliest work in the field hypothesized that ictal ischemic cell damage was associated with vasospasms during seizures that caused local pockets of hypoxia [Pfleger, (1880) Alg. Z. Psychiatrie 36:359-365; Spielmeyer, (1930) Arch. Neurol. Psychiatry 23:869-875].

This vascular ischemia hypothesis was contradicted by later observations by neurosurgeons in which febrile epileptic seizures exhibit hyperemia: in fact, the venous blood supply within the focus became more reddish (more oxygenated) during ictal periods (as if to mean that ictal foci were highly oxygenated and not hypoxic) [Penfield, W. and H. Jasper (1954) Epilepsy and the functional anatomy of the human brain. London, Churchill; Haglund et al., (1992) Nature 358:668-671]. Hyperemia is associated with edema, which is expected to contribute to the swelling seen in ictal sclerosis.

The observation of hyperemia during interictal periods suggested that metabolic excitotoxicity hypotheses were needed to explain ictal ischemic cell death, because no frank ischemia was seen. However, it is possible that both hyperemia, causing edema-related necrosis, and ischemia, causing hypoxia-related necrosis, occur in response to the same ictal episode (even one that is visibly hyperemic by direct observation from the cortical surface vessels). This can happen when local blood flow is shunted away from some capillaries due to ictal vasospasms of pericytes at the junctions of capillaries [Hirase et al. (2004) Neuroscience 128(1): 209-216; Hirase et al., (2004) Glia 46(1):95-100], and funneled to other The link between epilepsy and neuronal damage was first noticed almost 200 years ago [Bouchet et al., (1825) Arch Gén Med 9:510-542]. Since then, it has been established that seizures trigger a cascade of biochemical, anatomical, and functional changes that can, in some cases, lead to cell death and apoptosis [Cavanagh, et al., (1956). Br Med J 2:1403-1407; Meldrum, (2002) Prog Brain Res 135:3-11]. Neural events following seizures include neural injury, neurogenesis, gliosis, sprouting, and functional changes; it is currently a hot topic of debate whether these events occur in series as a cascade, or in parallel, or in some combination of serial and parallel processes [Cole et al., (2002) Prog Brain Res 135:13-23].

These various events are in general investigated in the context of primary excitotoxic mitochondrial damage resulting from calcium overload during ictal burst discharge [Meldrum et al., (1973) Arch Neurol 29(2):82-87; Olney et al., (1983) Brain Res Bull 10(5): 699-712; Olney et al., (1986) Adv Neurol 44:857-877; Sloviter, (1983) Brain Res Bull 10(5):675-697; Sloviter, (1986) Adv Exp Med Biol 203:659-671]. However, some of the earliest work in the field hypothesized that ictal ischemic cell damage was associated with vasospasms during seizures that caused local pockets of hypoxia [Pfleger, (1880) Alg. Z. Psychiatrie 36:359-365; Spielmeyer, (1930) Arch. Neurol. Psychiatry 23:869-875].

This vascular ischemia hypothesis was contradicted by later observations by neurosurgeons in which febrile epileptic seizures exhibit hyperemia: in fact, the venous blood supply within the focus became more reddish (more oxygenated) during ictal periods (as if to mean that ictal foci were highly oxygenated and not hypoxic) [Penfield, W. and H. Jasper (1954). Epilepsy and the functional anatomy of the human brain. London, Churchill; Haglund et al., (1992) Nature 358:668-671]. Hyperemia is associated with edema, which is expected to contribute to the swelling seen in ictal sclerosis.

The observation of hyperemia during interictal periods suggested that metabolic excitotoxicity hypotheses were needed to explain ictal ischemic cell death, because no frank ischemia was seen. However, it is possible that both hyperemia, causing edema-related necrosis, and ischemia, causing hypoxia-related necrosis, occur in response to the same ictal episode (even one that is visibly hyperemic by direct observation from the cortical surface vessels). This can happen when local blood flow is shunted away from some capillaries due to ictal vasospasms of pericytes at the junctions of capillaries [Hirase et al. (2004) Neuroscience 128(1): 209-216; Hirase et al., (2004) Glia 46(1):95-100], and funneled to other capillaries.

Thus, an overall hyperemic response can occur within the ictal focus (thus leading to edema), while at the same time having small volumes of tissue around the blocked capillaries that are ischemic (thus leading to pockets of hypoxia, and thus ischemic cell death due to frank lack of oxygen). These vasospasms that cause local hypoxia (and ischemic cell death) moreover shunt a surplus of oxygenated blood into the surrounding parenchyma, contributing to the general hyperemia (and resultant edema). It is therefore expected that some capillaries exhibit ischemia whereas others exhibit hyperemia during ictal periods [Siesjo et al., (1986) Adv Neurol 44:813-847; Tae et al., (2005) Neuroimage 24(1):101-110], as compared to blood flow in normal wild-type hippocampal capillaries.

The various factors that contribute to vision loss in age-related macular degeneration (AMD) are not well understood. AMD is a disease associated with aging that gradually destroys sharp, central vision. Central vision is needed for seeing objects clearly and for common daily tasks such as reading and driving.

AMD affects the macula, the part of the eye that allows us to see fine detail. In some cases, AMD advances so slowly that people notice little change in their vision. In others, the disease progresses faster and can lead to loss of vision in both eyes. AMD is the leading cause of vision loss in Americans 60 years of age and older. AMD occurs in two forms: wet and dry.

The dry form is characterized by yellow deposits in the back of the eye, called “drusen”. Dry AMD eventually leads to the break-down of the light-sensitive cells in the macula, and it gradually blurs central vision in the affected eye. As dry AMD advances, patients may see a blurred spot in the center of their vision. Over time, as less of the macula functions, central vision is gradually lost in the affected eye.

Wet AMD occurs when abnormal blood vessels behind the retina start to grow under the macula (neovascularization). These new blood vessels tend to be very fragile and often leak blood and fluid. The blood and fluid raise the macula from its normal place at the back of the eye. Damage to the macula occurs rapidly.

Most people with advanced AMD have the wet form. In all cases the wet form progresses after onset of the dry form.

A great deal of recent literature and patent reports have focused on the role of the carotenoids lutein and zeaxanthin in protecting the macula and use in slowing the progression of the disease. Thus, free radicals generated in the body during metabolism can damage the eye. Delicate tissues of the eye contain mainly polyunsaturated fatty acids that are vulnerable to damage by free radicals and oxidative stress.

In healthy eye tissues, large nutrients of antioxidants including lutein and zeaxanthin exist that can counter this damage. Lutein and zeaxanthin are yellow pigments that are highly concentrated in the macula. Various published studies suggest that intake of lutein, zeaxanthin or other carotenoids can lower eye diseases.

The yellow pigments of macula consisting of about equal amounts of lutein and zeaxanthin protect the macula form the damages of photoxidative effect of UV blue light. Lutein and zeaxanthin intake increases the serum level of lutein and zeaxanthin and improves the function of UV blue blocking and protection. Therefore those carotenoids are emerging as important nutrients for better health and prevention of disease. See, U.S. Pat. No. 7,179,930 to Bhaskaran et al. and the citations therein for a further discussion of carotenoids and AMD.

Ingestion of lutein and zeaxanthin does not appear to cure the disease, but may ameliorate its effects. The underlying cause of AMD has not been determined conclusively. However, one candidate is the change in blood flow that can result from and/or drive neovascularization within the retina in wet AMD. Abnormal blood flow causing either hypoperfusion or hyperperfusion or both can lead to swelling and/or oxygen starvation within the retina, as can also happen during a mini-stroke in the brain. The neovascularization leads to abnormal blood flow in the retina that in turn leads to death of retinal neurons.

One problem in diabetes patients is increased blood viscosity. High sugar levels leads to thick sticky blood, which causes degeneration of tissues of organs with fine vascular networks (i.e., kidneys, brain, heart, retinas, feet, etc) due to poor perfusion. Increased blood and/or serum viscosity over normal viscosity as is found in diabetic patients or animal models can lead to neurodegeneration.

Thus, McMillan [(1974) J. Clin. Invest 53(4):1071-1079] reported that the serum viscosity of diabetic patients has been found to be increased. The elevation averaged 8% above healthy subjects and 6% above nondiabetic patients. The serum viscosity elevation was greater when diabetic sequelae associated with microangiopathy were present. With such microangiopathy, the walls of very small blood vessels (capillaries) become so thick and weak that they bleed, leak protein, and slow the flow of blood.

No relation of serum viscosity to age, sex, obesity, duration of disease, or type of treatment was demonstrated. Serum total protein and glucose levels were found to be correlated with serum viscosity, and increases in their serum concentrations were observed in diabetes. Analysis demonstrated that their elevation did not explain either the viscosity increase or the difference in viscosity between diabetics with and without sequelae. [McMillan (1974) J. Clin. Invest 53(4):1071-1079.]

Intrinsic viscosity, abbreviated [η], is a concentration-independent solute property related to molecular shape. [η] Was found to be 7% higher in diabetic than in normal serum. The [η] difference accounted for at least half of the serum viscosity elevation. The rest of the increase was due to increased serum protein level and increased nonprotein solids, presumably glucose and lipid. Associated with increased [72 ] was a decline in albumin:globulin ratio and elevation of the acute phase reactant proteins, α₁-acid glycoprotein, α₁-antitrypsin, haptoglobin, and ceruloplasmin. Studies comparing diabetic and normal serum fractionated by using 21.5% sodium sulfate showed that changes in [η] were attributable to changes in serum protein composition rather than an inherent qualitative disturbance of protein present in one of the fractions. [McMillan (1974) J. Clin. Invest 53(4):1071-1079.]

The present invention provides one answer to the problem of neurotoxicity or neurodegeneration caused by abnormal blood flow, as is discussed below.

### BRIEF SUMMARY OF THE INVENTION

The present invention contemplates the treatment of neurotoxicity or neurodegeneration caused by abnormal blood flow, as can occur in wet age-related macular degeneration (AMD), epilepsy and diabetes, by administration of an effective amount of a blood flow regulatory drug, such as a drug already used to treat stroke, to a subject such as a patient in need thereof. This treatment is typically administered a plurality of times. Thus, the contemplated method treats abnormal blood flow to the organs such as the retina, brain and organs with fine vascular networks such as kidneys, heart, feet, and again brain and retina, and the like, and, stabilizes that blood flow, reducing hemorrhage and leaking of the blood vessels and thereby decreasing neuronal death.

### BRIEF DESCRIPTION OF THE FIGURES

In the drawings forming a portion of this disclosure,

FIGS. 1-3 are photomicrographs that show a combined intrinsic optical signal macroscopy and two-photon microscopy preliminary experiment we conducted in the olfactory bulb of OMP-GFP mice [Mombaerts et al., (1996) Cell 87(4): 675-686], whose olfactory neurons intrinsically make green fluorescent protein (GFP), and thus glow green.

FIG. 1 in three panels (FIGS. 1A-1C) shows functional activation of hexanol using intrinsic optical signal macroscopy, and targeting of a hexanol glomerulus for further analysis. These figures demonstrate the proof-of-concept and feasibility of mapping the mouse brain for specific function, followed by targeted functional microscopic blood flow analysis with two-photon imaging. FIG. 1A shows a green light (605 nm) reflectance photograph of left mouse olfactory bulb. Notice pronounced vascular artifact. Horizontal extent of image: about 2 mm. FIG. 1B illustrates an orange light (635 nm) image of the same area. Notice decreased vascular artifact. FIG. 1C is an IOSM image of hexanol activation (ratio of activated:non-activated bulb at 635 nm). Arrow marks hexanol sensitive glomerulus.

FIG. 2 in five panels (FIGS. 2A-2E) shows 3-dimensional subsurface analyses of the targeted glomerulus and microvasculature, with specific targeting of a capillary for subsequent blood flow analysis during functional activation with hexanol.

FIG. 3 contains three panels (FIGS. 3A-3C) that illustrate two-photon microscopic linescans of capillary blood flow analysis of baseline and functionally correlated blood flow responses. FIG. 3A shows a raw two-photon microscopic linescan of capillary denoted in FIG. 2E. The Y-axis denotes fluorescence along the length of a capillary during a single linescan, whereas the X-axis denotes time (repetitive linescans), with time increasing rightwards (2 msec per line). Black lines indicate red blood cells (RBCs). A shift in vertical position of black lines over time indicates movement of RBCs. FIG. 3B shows results of use of a custom RBC detection/digitization algorithm. Alternating RBCs are colored in green/blue. Stripe slope indicates velocity, (horizontal means RBC is stationary, vertical means it's moving very fast), whereas number of RBCs in a given column indicates Density. FIG. 3C provides V and D measurements of red blood cells through time (smoothed with 1 second median filter). Downward velocities indicate movement to the left, upward to the right. The rapid acceleration of red blood cells in the middle of the trace is in response to the onset of hexanol odor (onset time marked with cyan line in (FIG. 3C)). Note that V and D odor responses are not correlated in this sample, showing the importance of quantified analysis. Studies were also conducted in rats to compare blood flow responses within 2 (or more) differently tuned glomeruli (data not shown).

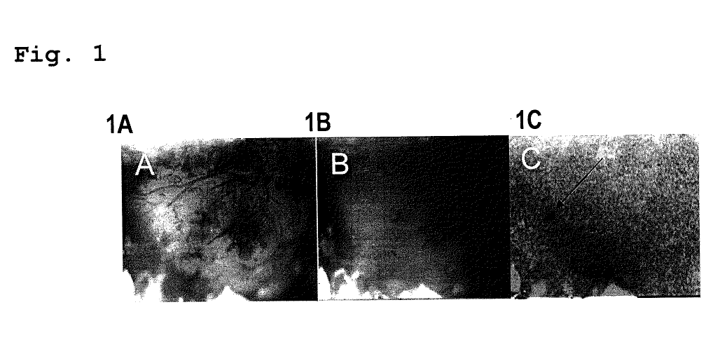

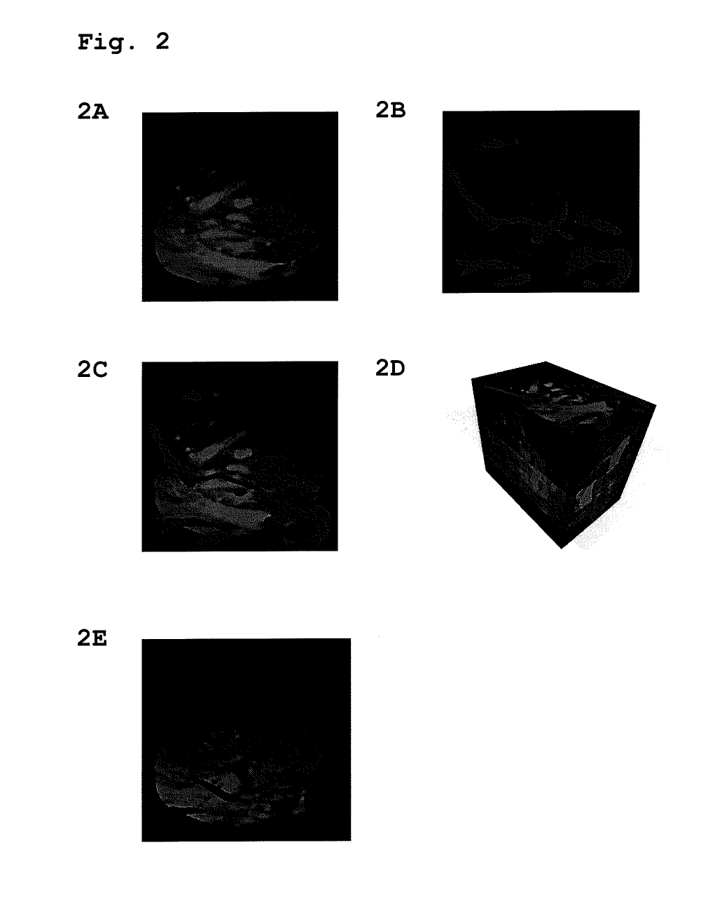

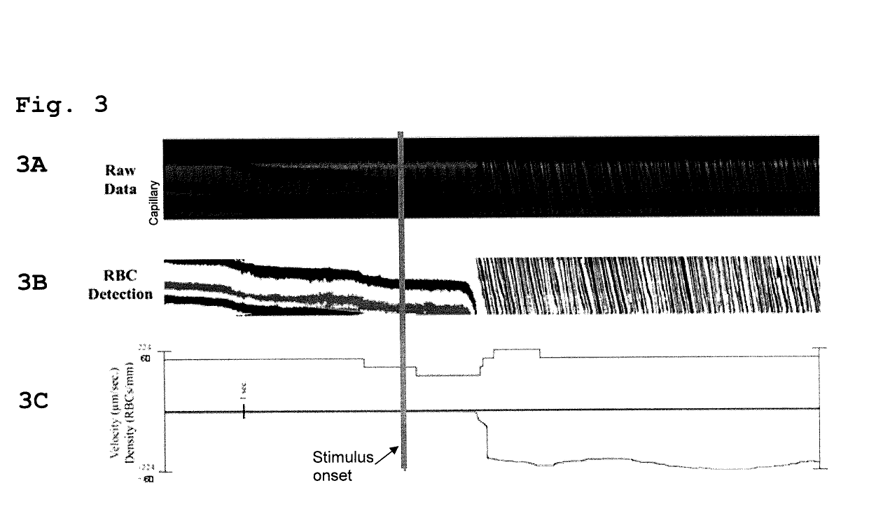

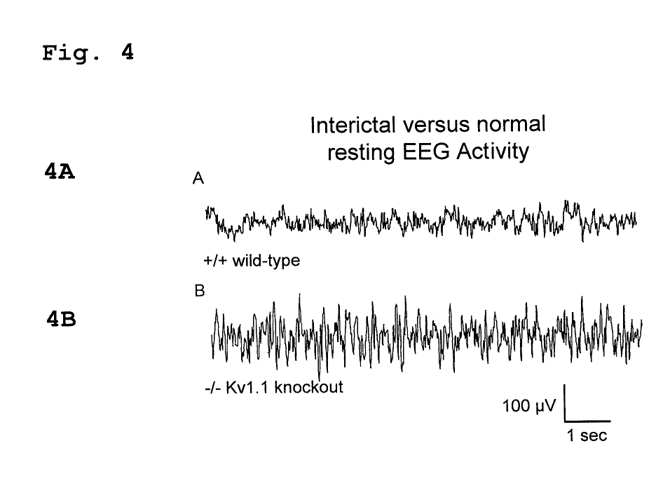

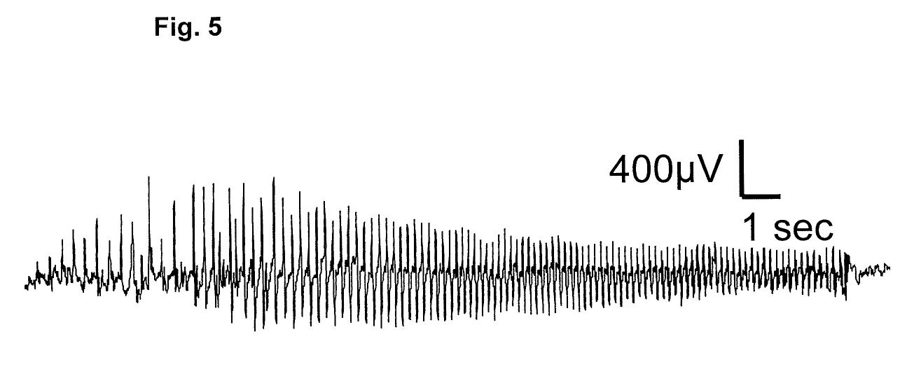

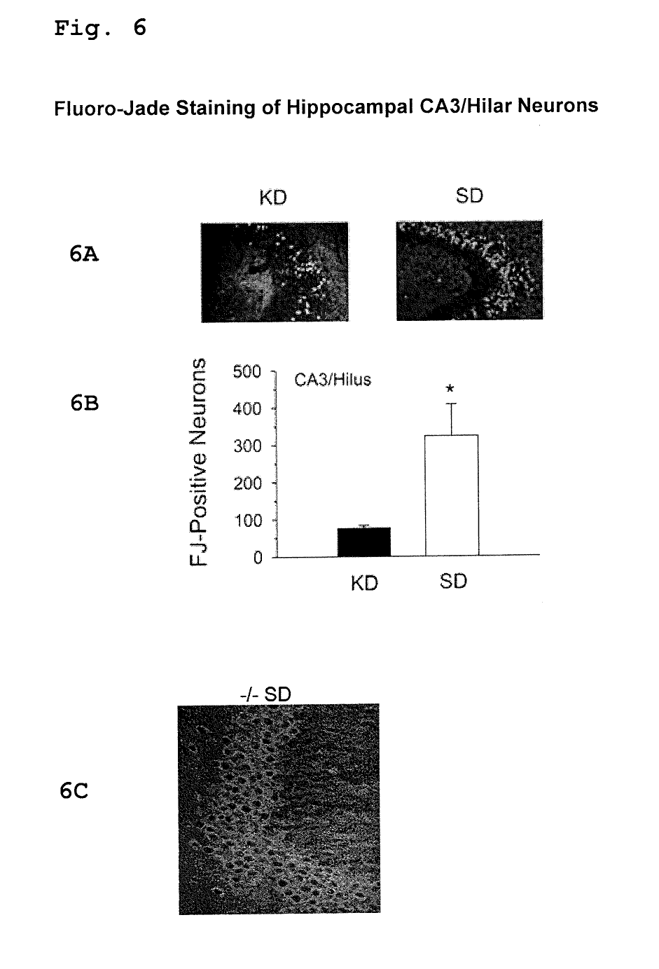

Fluorescein-labeled dextrans were injected into the bloodstream, and the activated glomerulus was imaged using two-photon laser scanning microscopy (FIG. 2). By capturing the red photons emitted with a red-filtered photomultiplier (photon detector) blood flow velocity and volume in rats and mice were recorded (FIG. 3). Using OMP-GFP mice [Mombaerts et al., (1996) Cell 87(4): 675-686], the neural structure of the glomeruli using a green-filtered photomultiplier could be simultaneously viewed. This technique permitted knowledge of exactly which capillaries inside the glomerulus had caused the macroscopic blood flow signal in FIG. 2C.

FIGS. 4 and 5 show EEG recordings from wild-type mice and also Kv1.1 knockout mice. The results suggest that interictal levels of activity may be sufficient, even in lieu of ictal activity, to cause neural damage through either blood flow- or non-blood flow-related factors. FIG. 4A illustrates a wild-type normal EEG recording. FIG. 4B shows an interictal EEG recording from Kv1.1 knockout mouse. The striking difference in physiological parameters suggests that interictal activity may be sufficient, even in the absence of ictal activity, to elevate blood flow to abnormal levels and/or to cause neural degeneration through either blood flow- or non-blood flow-related factors.

FIG. 5 shows the EEG activity of a Kv1.1 mouse recorded during an ictal episode.

FIG. 6 is a illustrates FluoroJade B histological staining in Kv1.1 knockout mice that illustrates that such staining is an effective means to assess neural degeneration and its amelioration by various treatments (in this case, the effect of the ketogenic diet in the hippocampus).

The present invention has several benefits and advantages. One benefit is that its method provides a new treatment for serious illnesses.

An advantage of the invention is that a contemplated treatment typically utilizes a medication already shown to be safe for human use, albeit for one or more other conditions, so that the risks and dangers often associated with the use of a new medication are be minimized.

Another benefit of the invention is the realization of a hitherto unappreciated cause of neurodegeneration that now known, can lead to still further treatment modalities.

Still further benefits and advantages of the invention will be apparent to the skilled worker for the discussion that follows.

### DETAILED DESCRIPTION OF THE INVENTION

A treatment for abnormal blood flow to one or more organs such as the eye, brain and previously noted organs with fine vascular networks is contemplated. This treatment ameliorates the disease state that can lead to neurodegeneration as in AMD, epilepsy and diabetes, respectively, by attacking its cause.

This method contemplates administration of a blood flow regulating effective amount of a blood flow regulatory drug to a subject exhibiting abnormal blood flow such as a human patient that is in need of such treatment, such as a subject having AMD, epilepsy or diabetes. This treatment reduces the cumulative degenerative effects of AMD, epilepsy and diabetes by utilizing a therapy already developed to protect patients such as stroke victims from ischemic cell death.

Thus, this treatment method addresses neural degeneration, the most insidious effect of abnormal blood flow (e.g., hypoperfusion or hyperperfusion or both), that develops within the course of the disease. In AMD, it is retinal cell death that leads to the blindness associated with AMD, whereas in epilepsy it is death of the brain cells themselves. In diabetes, death occurs in nerves as well as adjacent tissues.

Excessive magnitudes of local hyperperfusion and/or hypoperfusion in abnormal blood vessels can contribute, or even be a major cause, of this debilitating problem. Thus, hyperemia, causing edema-related necrosis and hypoperfusion, causing ischemia-related necrosis, can both occur. Both types of necrosis can occur because local blood flow can be regulated by smooth muscles and pericytes on blood vessels, and so it is possible to have an overall hyperemic response leading to edema, while at the same time having small volumes of tissue shut off to blood flow, thus leading to ischemia.

It is contemplated that the administration of the blood flow regulatory drug will be carried out a plurality of (multiple) times. Depending upon the patient, severity of the condition treated and the treating agent used, it is likely that that administration will be undertaken one to four times daily to weekly for the remainder of the recipient subject's life.

### Blood Flow Regulatory Drugs-Treating Agents

Several classes and specific treating agents (drugs, medications or medicaments) are contemplated for use. The drugs can be used alone or one or more drugs from each category can be used together. These classes of agents include anticoagulants and vasodilators, and mixtures thereof, both of which can be subdivided further.

For example, anticoagulant drugs include heparin, an NSAIDs such as aspirin and naproxen, as well as thrombolytic agents such as tissue plasminogen activator (tPA), streptokinase and urokinase that are thrombolytic agents (clot-dissolving enzymes).

Vasodilators are also useful. Illustrative vasodilating agents include organonitrates as are discussed hereinbelow, molsidomine, linsidomine chlorhydrate and S-nitroso-N-acetyl-d,l-penicillamine (“SNAP”); long and short acting α-blockers such as phenoxybenzamine, dibenamine, doxazosin, terazosin, phentolamine, tolazoline, prazosin, trimazosin, alfuzosin, tamsulosin and indoramin; ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; nimodepine, pinacidil, cyclandelate, dipyridamole and isoxsuprine; chlorpromazine; haloperidol; yohimbine; trazodone, vasoactive intestinal peptides and mixtures thereof. Prostaglandin E₁, an organonitrate and phentolamine are particularly preferred vasoactive agents for use in conjunction with the present method.

Organonitrate compounds are nitric oxide precursor vadodilators that can be co-administered (co-formulated) or administered separately in conjunction with another vasodilator or other drug. Illustrative organonitrates or nitric oxide precursors include erythrityl tetranitrate (1,2,3,4-butanetetrol tetranitrate), isosorbide dinitrate, nitroglycerin, pentaerythritol tetranitrate, isosorbide mononitrate and nicorandil [N-2-nitroxy)ethyl]-3-pyridinecarboxamide].

An organonitrate compound or other vasodilator is used in a vasodilating effective amount. Methods for measuring vasodilation using an organonitrate or other vasodilator are well known as are vasodilating amounts for internal use by oral or buccal administration, as such compounds are commercially available and approved for such uses by governmental bodies of many countries including the US FDA.

Additional vasodilators include medications otherwise useful in treating erectile disfunction and include oral phosphodiesterase inhibitors such as sildenafil citrate (Viagra®), tadalafil (Clalis®) and vardenafil (Levitra®) that inhibit PDE5, and milrinone (Primacor®) and inamrinone (Inocor®) that inhibit PDE3 and are otherwise useful as an ionotropic agent in heart failure patients.

A subject to which or whom a blood flow regulatory drug compound composition is administered can be and preferably is a human, but can also be an ape such as a chimpanzee or gorilla, a laboratory animal such as a monkey, rat, mouse or rabbit, a companion animal such as a dog, cat, horse, or a food animal such as a cow or steer, sheep, lamb, pig, goat, llama or the like.

A contemplated composition is administered to a subject in need of the medication at a blood flow regulatory effective dosage level. That level differs among the several medications contemplated as is well known for each medication. Illustrative effective dosages for the exemplary medications discussed above can be found in the Physician's Desk Reference, a yearly publication of Thomson Healthcare, as well as in texts such as Alfonoso R. Gennaro. Remington: The Science and Practice of Pharmacy, 20th ed., Lippincott Williams & Wilkins, Baltimore, Md. (2000) (formerly known as Remington's Pharmaceutical Sciences), and Goodman & Gilson's The Pharmacological Basis of Therapeutics, (9th ed.), McGraw-Hill, New York (1996). The amount of a particular medication can also vary depending on the recipient's age and weight as is well-known. Similar concentrations of blood flow regulatory drug compound medication or medicament) that can be provided by a liquid suspension for oral administration or a liquid composition for injection are also useful in providing a desired plasma or serum concentration.

Preferably, a contemplated pharmaceutical composition is in unit dosage form. In such form, the composition is divided into unit doses containing appropriate quantities of the active urea. The unit dosage form can be a packaged preparation, the package containing discrete quantities of the preparation, for example, packeted tablets, capsules, and powders in vials or ampules. The unit dosage form can also be a capsule, cachet, or tablet itself, or it can be the appropriate number of any of these packaged forms.

### Assays for Treatment

Assays for efficacy in human patients with diabetes and animal models for diabetes are readily accomplished. For example, one would know that an animal model of diabetes got better because one can periodically invasively test target tissues (i.e., brain, kidneys and the like) to examine the amount of cell death (as compared to age matched normals). In humans, the positive outcome would be known by an increased lifespan and statistical decrease in the occurrence of diabetic pathologies in the treated population. The medication is administered for the remainder of the patient's life time, only to be stopped if the diabetes was cured (or in the event of unfortunate drug interactions/side effects).

1. Confocal Fiber-Optic Microscopy of Blood Flow

Intrinsic imaging of ictal blood flow permits tracking of the expanding ictal focus across the surface of the brain area. Blood flow responses caused by ictal activity have been measured with intrinsic optical imaging in humans [Haglund et al. 1992 Nature 358:668-671)], monkeys Haglund et al., (1998) Society for Neuroscience 28th Annual Meeting, Los Angeles, Calif., Society for Neuroscience Abstracts], ferrets [Schwartz et al., (2001) Nat Med 7(9):1063-1067], and rats [Suh et al., (2005) J. Neuroscience 25(1):68-77], and have been suggested as potential contributors (in addition to excitotoxicity) to the chronic neural damage seen in epilepsy [Siesjo et al., (1986) Adv Neurol 44:813-847; Tae et al., (2005) Neuroimage 24(1):101-110].

The invention of fluorescence microscopy was a major leap forward for microscopic analysis. It permitted the illumination of tissue with one color of light (the excitation wavelength), while imaging only the fluorescently emitted photons of a different color. For biological specimens, the main problem with fluorescent imaging is that a fluorophore, by its quantum mechanical nature, is excited by a high-energy (bluish) photon and emits a lower energy (reddish) photon. This is a problem because brain tissue tends to absorb bluish photons, which are highly energetic, and which therefore tend to quickly cause severe photodamage (i.e., in the form of heat damage and free radicals) when illumination strengths were high enough to permit deep (over ˜100 microns) imaging.

The development of in vivo fiber-optic-based confocal microscopy has made possible the measurement of flow in deep capillaries [Denk et al., (1994) J Neurosci Methods 54(2):151-162; Kleinfeld et al., (1998) Proc Natl Acad Sci USA 95(26):15741-15746; Helmchen et al., (2001) Neuron 31(6):903-912; Chaigneau et al., (2003) Proc Natl Acad Sci USA 100(22):13081-13086; Larson et al., (2003) Science 300(5624): 1434-1436; Hirase et al., (2004) Neuroscience 128(1):209-216; Hirase et al. (2004), Glia 46(1):95-100; Schaffer et al., (2006) PLoS Biol 4(2):e22]. Because of the use of a fiber-optic, the use of a blue laser is possible because the objective is literally positioned within 100 microns of the tissue of interest, no matter how deep. With this technique, one injects a fluorescent dye (fluorescein dextrans, or quantum dots, for example) into the blood stream, in which the serum takes up the dye, but the red blood cells do not. Confocal microscopy then images the microvasculature by running a cannulated fiber-optic to the tissue of choice, irrespective of depth, and red blood cells can be counted (which appear as dark spots) as they flow through a capillary. This count measured over time reveals the flow dynamics, and these dynamics are compared during electrographic seizures in the hippocampus.

The change in blood flow dynamics after normal activation of the somatosensory cortex and olfactory bulb of rodents has been measured [Kleinfeld et al., (1998) Proc Natl Acad Sci USA 95(26):15741-15746; Helmchen et al., (2001) Neuron 31(6):903-912; Chaigneau et al., (2003) Proc Natl Acad Sci USA 100(22):13081-13086]. Hirase and colleagues measured blood flow changes within cortical bicuculline seizure loci in rodents with two-photon microscopy [Hirase et al., (2004) Neuroscience 128(1):209-216; Hirase et al. (2004), Glia 46(1):95-100], but it is not well established that the cortex is relevant to the study of ictal neural damage, and no normal blood flow measures in freely moving animals were assessed (therefore one cannot know if the seizure blood flow was abnormal). These potential shortcomings are addressed by targeting individual hippocampal capillaries and assessing blood flow as a function of ictal versus interictal flow in epileptic Kv1.1 knockout mice, as compared to blood flow in hippocampal capillaries of resting and active wild-type littermates.

2. The Kv1.1 Mouse Model System in Epilepsy Research

Rodents are excellent models for research in epilepsy [Ekstrand et al., (2001) J Comp Neurol 434(3):289-307], chronic neural degeneration [Kotloski et al., (2002) Prog Brain Res 135: 95-110] and ictal [Hirase et al., (2004) Neuroscience 128(1):209-216; Hirase et al., (2004) Glia 46(1):95-100] and normal blood flow [Chaigneau et al., (2003) Proc Natl Acad Sci USA 100(22):13081-13086]. Epilepsy research in the mouse is especially powerful because of the availability of transgenic strains relevant to human epilepsy.

The Kv1.1 knockout mouse (Kcna-1 null) is utilized herein as illustrative. This mouse lacks a delayed-rectifier voltage-gated potassium channel α-subunit protein [Smart et al., (1998) Neuron 20(4):809-819]. This mutation makes the Kv1.1 knockout mouse epileptic, and highly clinically relevant. Kv1.1 knockouts furthermore have similar histological indication of hippocampal neural damage as in humans, and the Kcna-1 gene is the only epilepsy gene in a developmental animal model that has a homologue in a human epileptic condition [Zuberi et al., (1999), Brain 122(Pt 5):817-825].

At birth, Kcna1-null mutants have no gross developmental defects and are virtually indistinguishable from littermates. However, by the third to fourth postnatal week, Kcna1-null mice exhibit abnormal behaviors consisting of episodic eye blinking, twitching of vibrissae, forelimb paddling, periodic arrest of motion, and hyperstartle responses. Recurrent spontaneous seizures are then observed, usually before the end of the first postnatal month. These seizures possess many of the characteristics of limbic system seizures in other rodent models (e.g., rearing, forelimb clonus), suggesting that structures such as the hippocampus may play an important role in their pathogenesis.

Nearly all Kcna1-null mice generated in the C3HeB/FeJ background do not ordinarily survive much beyond postnatal day 60. It is unclear what the precise cause(s) of the high mortality is (are), but several knockout mice have been observed to experience either a severe acute seizure or status epilepticus lasting several hours immediately prior to death. Control mice do not die at this young age.

Histological assessment of the brains of 2-3 month-old Kcna1-null mice reveals characteristic morphological changes associated with chronic epilepsy, most notably in the hippocampus. There is hilar cell loss and increased GFAP (glial fibrillary acidic protein) expression in areas where there is normally high expression of the Kv1.1 protein, particularly the CA3 and dentate regions of the hippocampus. In addition, there is evidence of striking synaptic reorganization (i.e., mossy fiber sprouting in the inner molecular layer of the dentate gyrus as demonstrated with Timm staining) first noted after 5-6 weeks of postnatal life.

3. Assays in Model Ictal Systems

Ictal hyperemia and/or ischemia leads to neuronal damage. The contribution of ictal blood flow to neural damage (assessed with Fluorojade B staining) is determined by measuring the neural damage in the hippocampus, as a function of systemic treatment with a blood flow suppressor (e.g., 7-nitroindazole; 50 mg/kg IP) and a blood flow enhancer (e.g., acetazolamide; 30 mg/kg IP). Suppressing blood flow modulation with 7-nitroindazole results in less neural damage, and enhancing blood flow with acetazolamide results in increased neural damage.

Hippocampal electrographic seizure activity in Kv1.1 mutant mice produces abnormally high fluctuations of blood flow (hyperemia and ischemia) within individual capillaries, as compared to blood flow during inter-ictal states in the same capillaries. The blood flows are assayed in freely moving Kv1.1 mutant mice, as a function of behavioral state (as assessed by video-EEG), and as compared to hippocampal capillary blood flow in normal C3Heb/FeJ littermates during the same behavioral states. These mice illustrate that ictal blood flow is abnormal in comparison to blood flow in wild-type mice. That ictal blood flow is pathological at the level of the capillary, resulting in periods of both transient ischemia and hyperemia.

Ictal hyperemia and/or ischemia leads to neuronal damage. The contribution of ictal blood flow to neural damage is assessed with FluoroJade B staining to measure the neural damage in the hippocampus, as a function of systemic treatment with a blood flow suppressor (7-nitroindazole; 50 mg/kg IP) and enhancer (acetazolamide; 30 mg/kg IP). Suppressing blood flow with 7-nitroindazole results in less neural damage, whereas enhancing blood flow with acetazolamide results in increased neural damage.

Neural damage nearby to specific capillaries that have been scanned during the various conditions is localized by the microscope's cannula track (coated in Di-I [DiCarlo et al., (1996) J Neurosci Methods 64(1): 75-81], and is assessed with FluoroJade B histology (as is discussed below). The hippocampal tissue from the study side of the brain is compared for damage so induced to hippocampal tissue from the other hemisphere in the same sections, to confirm that the implantation of the fiber-optic microscope did not itself cause significant damage (this is confirmed by comparing Kv1.1 mice to normal littermates, both if which undergo equivalent microscopic measurement protocols).

Neural damage is greater in ictal conditions than in inter-ictal conditions or in wild-type mice. Moreover, comparing wild-type to Kv1.1 mice, one can determine the propensity of epileptic tissue for neural damage, as a function of activity level and ictal state (Table-1, groups 1-3).

TABLE 1

| | # | | Blood Flow | Pharmacological |
|---|---|---|---|---|
| Group | Mice | Genotype | Measurements | Treatment |
| 1 | 8 | −/− | Inter-ictal | Sham |
| | | | (freely moving) | |
| 2 | 8 | +/+ | Freely moving | Sham |
| 3 | 8 | −/− | Ictal | Sham |
| 4 | 8 | −/− | Inter-ictal | acetazolamide |
| | | | (freely moving) | |
| 5 | 8 | +/+ | Freely moving | acetazolamide |
| 6 | 8 | −/− | Ictal | acetazolamide |
| 7 | 8 | −/− | Inter-ictal | 7-nitroindazole |
| | | | (freely moving) | |
| 8 | 8 | +/+ | Freely moving | 7-nitroindazole |
| 9 | 8 | −/− | Ictal | 7-nitroindazole |

Total mice for use: 72 (48 Kv1.1 mice; from an estimated 24-32 litters, 6-8 total mice per litter, Kv1.1 homozygous mutants are born with a 25% probability).

This study provides the mechanistic basis of the contribution of abnormal blood flow in long-term damage in epilepsy [Siesjo et al., (1986) Adv Neurol 44:813-847; Tae et al.' (2005) Neuroimage 24(1):101-110]. Ictal blood flow contributes to neural damage in seizure foci.

There is growing evidence that brief seizures, historically thought to not cause neural damage, may in fact cause small amounts of subclinical damage with each episode (see FIG. 6); the damage from these episodes may build up cumulatively to produce behavioral and cognitive deficits [Bengzon et al., (1997) Proc Natl Acad Sci USA 94(19):10432-10437. The preliminary data (FIG. 6C) indicate that there is overall less FluoroJade B intensity in the principal cells during naturally occurring seizures than in seizures driven by kainate injections. This is not surprising, because status epilepticus induced by kainate is more severe an insult. Furthermore, the level of mean red blood cell flux increase seen by previous two-photon microscopy studies of ictal blood flow [Hirase et al. (2004) Glia 46(1):95-100] is roughly equivalent to peak transient flux increases caused by normal activation in other studies [Kleinfeld et al. (1998), Proc Natl Acad Sci USA 95(26):15741-15746; Chaigneau et al. (2003), Proc Natl Acad Sci USA 100(22):13081-13086]. This indicates that peak ictal blood flow is higher than peak normal blood flow.

Status epilepticus can also be induced in mouse subjects with kainate (confirmed in FIGS. 6A-6B) to produce statistically significant effects of neural damage in this mouse population) [Kotloski et al., (2002) Prog Brain Res 135:95-110]. Full seizure models of blood flow exhibit continual discharge from neurons and are likely to create an increased metabolic load [Nehlig et al., (1995) J Cereb Blood Flow Metab 15(2):259-269; Andre et al. (2002), Epilepsia 43(10):1120-1128; Arzimanoglou et al., (2002) Epileptic Disord 4(3):173-82].

A three-stage injection protocol is employed to induce status epilepticus with kainate. Mice are given 10 mg/kg (in pH 7.4 saline) of kainate subcutaneously, after which they begin electroclinical seizure activity. An additional 5 mg/kg is administered 30 minutes after behavioral seizure initiation, followed by another 5 mg/kg dose sc after another 30 minutes. This protocol has previously provided a high survivability rate (greater than 90%) in the C3Heb/FeJ strain of Kv1.1 knockouts, and to moreover sustain the status for at least a 2-hour duration. Behavioral seizure scoring is conducted using a modified Racine scale, and cumulative seizure scores are generated for each animal (a seizure score is given every minute, with the highest numerical value based on the Racine scale; then the cumulative score is generated by adding all the individual scores for each minute of seizure activity).

In groups 4-6 in Table-1 above, seizures are studied in Kv1.1 mutants that have been injected with acetazolamide (30 mg/kg IP) [Herson et al., (2003) Nat Neurosci 6(4):378-383], which invokes increased blood flow. A possible side effect of acetazolamide is that it may decrease the potency, or even abolish the formation of seizures due to its anti-convulsant effect [Resor et al., (1990) Neurology 40(11):1677-1681]. However, because the purpose of the study is to determine the contribution of blood flow to neural damage, the anti-convulsant side effect of acetazolamide can be beneficial. Because acetazolamide acts to reduce seizures [Resor et al., (1990) Neurology 40(11):1677-1681] any damage above and beyond that found without acetazolamide is even more likely to be caused by increased blood flow. If, instead, the epileptogenesis of Kv1.1 mice overcomes the anti-convulsant effect of acetazolamide, the dual contribution of seizures and enhanced blood flow can be determined.

Epileptic Behavioral Scoring

Each observed seizure is scored with the scale: 0, normal behavior (active or sleep); stage 1, motor arrest with or without facial twitches/chewing; stage 2, head bobbing and/or jerking; stage 3, forelimb clonus; stage 4, rearing; stage 5, rearing and falling. Blood flow increases are examined as a function of seizure intensity. Neural damage assessments are not possible to correlate to seizure intensity because they are assessed through examination of FluoroJade B staining of postmortem tissue, which reflects the neural damage accumulated over the entire scope of the animal's life (including before the experiment started).

Fiber-Optic Confocal Microscopy of Blood Flow within the Hippocampus of Mice

The mice are anesthetized [IP injection of a cocktail of Diazepine (5 mg/mL), Fentanyl (50 μg/mL) and Medetomidine (1 mg/mL)], with periodic maintenance of fentanyl/medetomidine [Mainen et al. (2000). Society for Neuroscience 30th Annual Meeting, New Orleans, La.]. Skin over area the bregma area of the skull is first removed, and fastened to a chamber to the skull. Inside the chamber a craniotomy is created and a durotomy exposes the brain. A cannula (that serves as the guide-tube for the fiber-optic of the microscope) is advanced down to the hippocampus. The cannula is stained with a lipophilic carbocyanine dye such as Di-I (1,1′-dioctadecyl-3,3,3′,3′-tetramethylinso-carbocyanine perchlorate) {Sparks, 2000 #11541; Honig, 1986 #11536; Mufson, 1990 #11540} and its tip within the tissue serves to indicate the area of interest for the FluoroJade B staining. Cell membranes constitute a convenient target for lipophilic dyes, whether cells are loaded live or fixed. Such lipophilic dyes can be tolerated by most cells at high concentrations, and these dyes can laterally diffuse within the membrane—in the process staining the entire cell, even if the dye is applied locally. One of the advantages of using a carbocyanine tracer such as Di-I is its slow diffusion time (days to weeks; 6 mm/day in live tissue and more slowly in fixed specimens) and limited penetration (within a few mm of the injection site); this makes it an ideal cannula-tract tracing dye {Bartheld, 1990 #11542}.

A red fluorescent dye (5% fluorescein isothiocyanate dextrans; 2MD; 1 mL/kg) is then injected IV into the blood serum, and capillaries within the hippocampus are targeted with 2D confocal microscopy. Appropriate capillaries are line-scanned to increase temporal fidelity of the blood flow dynamics.

Pharmacological Treatments with Acetazolamide and 7-Nitroindazole

Ten minutes before imaging blood flow, drugs will be administered by IP injection at a volume of 0.01 ml per g of body weight. Acetazolamide will be dissolved in 0.9% NaCl (pH 9.4 with NaOH) and administered at a dosage of 30 mg/kg (Herson, Virk et al. 2003). 7-Nitroindazole is suspended in a 1% solution of Tween 80 (Sigma, St. Louis, Mo., USA), and administered at a dosage of 50 mg/kg (Borowicz, Kleinrok et al. 2000).

Fluorojade B Histological Processing

FluoroJade B histological processing (as in FIG. 6) follows the methods set out previously by [Schmued et al., (2000) Brain Res 874(2):123-130]. Briefly, animals are anesthetized with pentobarbital (100 mg/kg IP), and given transcardiac perfusion with 300 ml of 0.1 M neutral phosphate buffered 10% formalin (4% formaldehyde). Animals are decapitated, after arrest of respiration and heart beat, and the brains quickly dissected. The brains are post-fixed overnight (about 18 hours) in the same fixative solution plus 20% sucrose. The brain is sliced into 10-micron sections, collected in 0.1 M neutral phosphate buffer, mounted on 2% gelatin coated slides and dried.

The slides are immersed in 1% sodium hydroxide in 80% alcohol (20 ml of 5% NaOH added to 80 ml absolute alcohol) for 5 minutes, followed by 2 minutes in 70% alcohol and 2 minutes in distilled water. The slides are transferred into a solution of 0.06% potassium permanganate for 10 minutes, while being shaken to ensure consistent background suppression between sections. The slides are then rinsed in distilled water.

The staining solution is made with 0.01% stock solution of FluoroJade B (Histo-Chem. Inc., Jefferson Ark.) in distilled water (final dye concentration of 0.0004%). After 20 minutes in the staining solution, slides are rinsed and dried. The dry slides are cleared by immersion in xylene for at least 1 minute before coverslipping with DPX (Fluka, Milwaukee Wis.; or Sigma Chem. Co., St. Louis Mo.). The tissue representing the hippocampus is then examined using epifluorescence microscopy with blue (450-490 nm) excitation light: the position of the imaging focus is localized by the red Di-I track from the cannula, and neuronal damage is assessed by FluoroJade B staining colored green.

Stereological Analysis of Neural Damage Assessed with Fluorojade B Staining

Assessment of neural damage follows established stereological methodology [Mouton, (2002) Principles And Practices Of Unbiased Stereology: An Introduction For Bioscientists, Baltimore and London, The Johns Hopkins University Press]. Briefly, every sequential 10-micron section containing the hippocampus is analyzed (approximately 24-30 sections). FluoroJade B stained cells are estimated following standard stereological counting procedures (Mouton, above). Sections containing the imaging site are localized by the presence of Di-I marked electrode tracks [DiCarlo et al., (1996) J Neurosci Methods 64(1):75-81]. Stereologer software is used to randomly assign, in an unbiased manner, a systematic grid of stereology disectors to optimize sampling and minimization of sampling error. FluoroJade B stained neurons are counted, following the disector principle, within the 500-micron radius of the tip of the fiber-optic confocal lens and an estimate of the number of degenerating neurons is established.

Statistical Analyses

For all analyses, the significance of differences among groups is set at p≦0.05. Differences in group means are assessed with ANOVA and post-hoc comparisons employ a 2-tailed t-test.

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A method for treatment of neurotoxicity or neurodegeneration caused by abnormal blood flow that comprises administration of an effective amount of a blood flow regulatory drug to a subject in need thereof.

2. The method according to claim 1, wherein said administration is carried out a plurality of times.

3. The method according to claim 1, wherein said subject is a human patient.

4. The method according to claim 1, wherein said blood flow regulatory drug is an anticoagulant, a vasodilator, or a mixture thereof.

5. The method according to claim 4, wherein said blood flow regulatory drug is an anticoagulant.

6. The method according to claim 5, wherein said anticoagulant is selected from the group consisting of heparin, an NSAID and a clot-dissolving enzyme.

7. The method according to claim 4, wherein said blood flow regulatory drug is a vasodilator.

8. The method according to claim 7, wherein said vasodilator is selected from the group consisting of an organonitrate, a phosphodiesterase inhibitor, an α-blocker, an ergot alkaloid, an antihypertensive agent, and prostaglandin E₁.

9. The method according to claim 7, wherein said vasodilator is prostaglandin E₁, an organonitrate or phentolamine.

10. The method according to claim 1, wherein said neurotoxicity or neurodegeneration is caused by wet age-related macular degeneration.

11. The method according to claim 1, wherein said neurotoxicity or neurodegeneration is caused by epilepsy.

12. The method according to claim 1, wherein said neurotoxicity or neurodegeneration is caused by diabetes.

13. A method for treatment of neurotoxicity or neurodegeneration caused by abnormal blood flow in a human patient having wet age-related macular degeneration that comprises administration of an effective amount of a blood flow regulatory drug that is an anticoagulant, a vasodilator, or a mixture thereof to said patient.

14. The method according to claim 13, wherein said administration is carried out multiple times.

15. A method for treatment of neurotoxicity or neurodegeneration caused by abnormal blood flow in a human patient having epilepsy that comprises administration of an effective amount of a blood flow regulatory drug that is an anticoagulant, a vasodilator, or a mixture thereof to said patient.

16. The method according to claim 15, wherein said administration is carried out multiple times.

17. A method for treatment of neurotoxicity or neurodegeneration caused by abnormal blood flow in a human patient having diabetes that comprises administration of an effective amount of a blood flow regulatory drug that is an anticoagulant, a vasodilator, or a mixture thereof to said patient.

18. The method according to claim 17, wherein said administration is carried out multiple times.
